(19) United States
(12) Patent Application Publication
(10) Pub. No.: US 2010/0061946 A1
(43) Pub. Date: Mar. 11, 2010

(54) COSMETIC FORMULATION HAVING GLUCOSYL GLYCERIDES AND SELECTED PERFUME SUBSTANCES

(75) Inventors: Cathrin Scherner, Norderstedt (DE); Rainer Kroepke, Schenefeld (DE); Ute Breitenbach, Hamburg (DE); Jens Treu, Norderstedt (DE); Werner Zilz, Halstenbek (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 12/515,165

(22) PCT Filed: Nov. 14, 2007
(86) PCT No.: PCT/EP07/09836

§ 371 Date: Aug. 31, 2009

(30) Foreign Application Priority Data
Nov. 21, 2006 (DE) 10 2006 056 320.4

(51) Int. Cl. A61K 8/06; A61K 8/60; A61Q 19/08

(52) U.S. Cl. 424/59; 514/25

(57) ABSTRACT

A cosmetic preparation which comprises one or more selected perfume substances and one or more glucosyl glycerides.

The present invention relates to a cosmetic preparation with selected perfume substances and one or more glucosyl glycerides.

The desire to appear beautiful and attractive is naturally rooted in man. Even if the beauty ideal has undergone changes over the course of time, the desire for a flawless outward appearance has always been the aim of human beings. The condition and the appearance of the skin is a significant part of a beautiful and attractive outward appearance.

In order for the skin to be able to fulfill its biological function fully, it needs regular cleansing and care. The cleansing of the skin serves to remove dirt, sweat and the residue of dead skin particles that provide an ideal breeding ground for germs and parasites of all kinds. Skin care products are used mostly for moisturizing and regreasing the skin. Active substances are often added thereto that regenerate the skin and, e.g., are designed to prevent and reduce its premature aging (e.g., the formation of fine lines, wrinkles).

Cosmetics normally contain a number of perfume substances, which are used to cover up unpleasant inherent odors of preparation constituents and to give the cosmetic the individual scent that is typical for the manufacturer. However, a number of perfume constituents are not entirely unproblematic from a dermatological point of view. In particular, limonene, citral, linalool, alpha-isomethylionone, geraniol and citronellol can in individual cases lead to skin irritation, redness and other intolerance reactions with users with particularly sensitive skin.

The object of the present invention was therefore to eliminate the disadvantages of the prior art and to develop perfumed cosmetic preparations that are friendlier to the skin.

The object is surprisingly attained through a cosmetic preparation containing a combination of
a) one or more perfume substances selected from the group 2-isobutyl-4-hydroxy-4-methyltetrahydropyran, 2-tert-pentylcylclohexylacetate, 3-methyl-5-phenyl-1-pentanol, 7-acetyl 1,1,3,4,4,6-hexamethyltetralin, adipic diester, alpha-amylcinnamaldehyde, alpha-methylionone, amyl C butylphenyl methylpropional cinnamal, amyl salicylate, amyl cinnamyl alcohol, anisic alcohol, benzoin, benzyl alcohol, benzyl benzoate, benzyl cinnamate, benzyl salicylate, bergamot oil, bitter orange oil, butylphenyl methylpropional, cardamom oil, cedrol, cinnamal, cinnamyl alcohol, citronellyl methylcrotonate, lemon oil, coumarin, diethyl succinate, d-limonene, ethyl linalool, eugenol, evernia furfuracea extract, evernia prunastri extract, farnesol, guaiacwood oil, hexyl cinnamal, hexyl salicylate, hydroxycitronellal, hydroxyisohexyl 3-cyclohexene carboxaldehyde, lavender oil, lime oil, linalyl acetate, mandarin oil, menthyl PCA, methyl heptenone, myristica oil, oil of rosemary, sweet orange oil, terpineol, tonka bean oil, triethylcitrate, and/or vanillin and
b) one or more glucosyl glycerides.

According to the invention embodiments of the present invention that are advantageous are characterized in that the preparation according to the invention contains the glucosyl glyceride(s) in a concentration of from 0.01 to 10% by weight, preferably in a concentration of from 0.05 to 6% by weight and particularly preferably in a concentration of from 0.1 to 3% by weight, respectively based on the total weight of the preparation.

Preparation according to one of the preceding claims, characterized in that the preparation contains one or more perfume substances in a total amount of from 0.005 to 0.5% by weight, based on the total weight of the preparation.

It is advantageous according to the invention if the preparation is free from (2-hydroxyethyl)urea.

Glucosyl glycerides of the formula

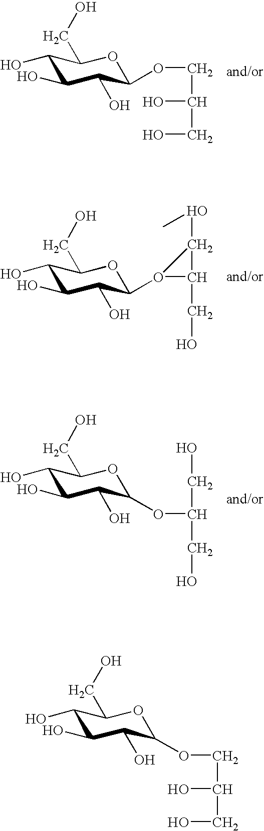

are advantageous within the scope of the present invention.

Although DE 195 40 749 and the unpublished DE 10 2005 023634, DE 10 2005 023635, DE 10 2005 023636, DE 10 2005 023637, DE 10 2005 023638, DE 10 2005 023639, DE 10 2005 023640, DE 10 2005 023641, DE 2006 034530.4 and DE 20 2006 011472.6 are described in the prior art, as well as JP 2004-331581, JP 2004-331576, JP 2004-331578, JP 2004-331579, JP 2004-331580, JP 2004-331582, JP 2004-331583, which are incorporated herein as reference, these documents could not show the way to the present invention.

Advantageously according to the invention the preparation according to the invention is present in the form of a gel, an emulsion, or a dispersion. It is preferred according to the invention thereby if the preparation according to the invention is present in the form of an emulsion.

In an embodiment of the present invention that is particularly preferred according to the invention the preparation is present in the form of an O/W emulsion.

In this case it is preferred according to the invention if the preparation contains one or more O/W emulsifiers selected from the group of the compounds glyceryl stearate citrate, glyceryl stearate, cetearyl glucoside, stearic acid and salts thereof, polyglyceryl-3-methylglycose distearate, ceteareth-20, PEG-40 stearate, PEG-100 stearate, sodium cetearyl sulfate.

These O/W emulsifiers according to the invention can be contained in the preparation advantageously according to the invention in a concentration of from 0.001 to 10% by weight and preferably in a concentration of from 0.1 to 7% by weight, based on the total weight of the preparation.

Another embodiment of the present invention that is preferred according to the invention is characterized in that the preparation is present in the form of a W/0 emulsion.

In the case of this embodiment it is preferred according to the invention if the preparation contains one or more W/O emulsifiers selected from the group of the compounds polyglyceryl-2-dipolyhydroxystearate, PEG-30 dipolyhydroxystearate, cetyl dimethicone copolyol, polyglyceryl-3 diisostearate.

These W/O emulsifiers according to the invention can be contained in the preparation in an advantageous manner according to the invention in a concentration of from 0.1 to 10% by weight and preferably in a concentration of from 0.2 to 7% by weight, based on the total weight of the preparation.

Advantageous embodiments of the present invention are characterized in that the preparation contains one or more further UV filters selected from the group of the compounds phenylene-1,4-bis-(2-benzimidazyl)-3,3′-5,5′-tetrasulfonic acid salts; 2-phenylbenzimidazol-5-sulfonic acid salts; 1,4-di(2-oxo-10-sulfo-3-bornylidenemethyl)benzene and salts thereof; 4-(2-oxo-3-bornylidenemethyl)benzene sulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]-disiloxanyl]propyl]phenol; 3-(4-methylbenzylidene)camphor; 3-benzylidenecamphor; ethylhexyl salicylate; terephthalidene dicamphor sulfonic acid; 4-(dimethylamino)benzoic acid (2-ethylhexyl)ester; 4-(dimethylamino)benzoic acid-amyl ester; 4-methoxybenzalmalonic acid di(2-ethylhexyl)ester; 4-methoxycinnamic acid (2-ethylhexyl)ester; 4-methoxycinnamic acid isoamyl ester; 2-hydroxy-4-methoxybenzo-phenone, 2-hydroxy-4-methoxy-4′-methylbenzophenone; 2,2′-dihydroxy-4-methoxybenzophenone; 2-(4′-diethylamino-2′-hydroxybenzoyl)benzoic acid hexyl ester, 4-(tert.butyl)-4′-methoxydibenzoylmethane; homomethyl salicylate; 2-ethylhexyl 2-hydroxybenzoate; 2-ethylhexyl 2-cyano-3,3-diphenylacrylate; dimethicodiethyl benzalmalonate; (3-(4-(2,2-bis-ethoxycarbonylvinyl)-phenoxy)propenyl)-methoxysilane/dimethylsiloxane copolymer; 2,4-bis-{[4-(2-ethylhexyloxy)-2-hydroxy]-phenyl}-6-(4-methoxyphenyl)-1,3,5-triazine (INCI: bis-ethylhexyloxyphenol methoxyphenyl triazine); dioctylbutylamidotriazone (INCI: diethylhexyl butamidotriazone); 2,4-bis-[5-1(dimethylpropyl)benzoxazol-2-yl-(4-phenyl)-imino]-6-(2-ethylhexyl)-imino-1,3,5-triazine with the (CAS no. 288254-16-0); tris(2-ethylhexyl) 4,4′,4″-(1,3,5-triazine-2,4,6-triyhriimino)trisbenzoate (also: 2,4,6-tris-[anilino-(p-carbo-2′-ethyl-1′-hexyloxy)]-1,3,5-triazine (INCI: ethylhexyl triazone); 2,4,6-tribiphenyl-4-yl-1, 3,5-triazine; merocyanine; titanium dioxide; zinc oxide in a concentration of from 0.01 to 40% by weight and preferably in a concentration of from 1 to 20% by weight based on the total weight of the preparation.

It is advantageous according to the invention if the preparation according to the invention is free from p-methylbenzylidene camphor.

The pigments (titanium dioxide, zinc oxide) can also be used advantageously in terms of the present invention in the form of commercially available oily or aqueous predispersions. Advantageously, dispersants and/or solubilizers can be added to these predispersions.

The pigments (titanium dioxide, zinc oxide) can advantageously be surface-treated (coated) according to the invention, wherein, e.g., a hydrophilic, amphiphilic or hydrophobic character is to be formed or retained. This surface treatment can consist in providing the pigments with a thin hydrophilic and/or hydrophobic inorganic and/or organic layer according to processes known per se. The various surface coatings can also contain water for the purposes of the present invention.

Inorganic surface coatings for the purposes of the present invention can comprise aluminum oxide (Al₂O₃), aluminum hydroxide Al(OH)₃, or aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), barium sulfate (BaSO₄) or iron oxide (Fe₂O₃). These inorganic surface coatings may be present on their own, in combination and/or in combination with organic coating materials.

Organic surface coatings for the purposes of the present invention can comprise vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may be present on their own, in combination and/or in combination with inorganic coating materials.

Embodiments of the present invention that are advantageous according to the invention are characterized in that the preparation according to the invention contains one or more compounds selected from the group of parabens, phenoxyethanol, ethylhexyl glycerin, 2-methylpropane-1,3-diol, butylene glycol, propylene glycol, which can be present therein in a total concentration of from 0.01 to 10% by weight based on the total weight of the preparation.

Advantageously according to the invention the preparation according to the invention contains as further ingredients one or more compounds selected from the group of the compounds folic acid, phytoene, D-biotin, coenzyme Q10, alpha-glucosylrutin, carnitine, polydocanol, natural and/or synthetic isoflavonoids, in particular genistein, flavonoides, carotenoids, creatine, creatinine, taurine, urea, ascorbic acid+derivatives oxygen, tocopherol+esters, dihydroxyacetone, 8-hexadecene-1,16 dicarboxylic acid, long chain and short chain hyaluronic acid (i.e., hyaluronic acid with an average molecular weight of from 1 million to 3 million Dalton, as well as 5000-1 million Dalton) and/or licochalcone A. Ingredients of this type can be contained respectively in an individual concentration of from 0.01 to 10% by weight, based on the total weight of the preparation. The use of carnitine is thereby particularly preferred according to the invention.

It is advantageous according to the invention if the preparation according to the invention additionally contains urea in a concentration of from 0.01 to 50% by weight, preferably in a concentration of from 0.1 to 20% by weight and particularly preferably in a concentration of from 1 to 15% by weight based on the total weight of the preparation.

In addition, the preparations according to the invention can advantageously also contain repellents for protection against flies, ticks and spiders and the like. For example, N,N-diethyl-3-methylbenzamide (trade name: “DEET”), dimethyl phthalate (trade name: Palatinol M, DMP), 1-piperidinecarboxylic acid-2-(2-hydroxyethyl)-1-methylpropylester and in particular ethyl 3-(N-n-butyl-N-acetylamino)propionate (available under the trade name Insekt Repellent®. 3535 from Merck) are advantageous. The repellents can either be used individually or in combination.

Moisturizers is the term used to describe substances or mixtures of substances which, following application or distribution on the surface of the skin, impart to cosmetic preparations the property of reducing the moisture loss by the horny layer (also called transepidermal water loss (TEWL)) and/or positively influencing hydration of the horny layer.

Advantageous moisturizers for the purposes of the present invention are, for example, glycerol, lactic acid and/or lactates, in particular sodium lactate, butylene glycol, propylene glycol, panthenol, biosaccharide gum-1, glycine soya, ethylhexyloxy glycerin, pyrrolidone carboxylic acid and hyaluronic acid. It further is in particular advantageous to use polymeric moisturizers from the group of polysaccharides which are soluble in water and/or swellable in water and/or form a gel in water. Particularly advantageous is, for example, a fucose-rich polysaccharide which is listed in Chemical Abstracts under the registry number 178463-23-5 and is available, for example, under the name Fucogel®1000 from SOLABIA S.A. Moisturizers can advantageously also be used as anti-wrinkle active ingredients for the protection from changes in the skin, as arise, for example, during skin aging.

It is advantageous for the purposes of the present invention if the preparation according to the invention contains one or more humectants in a total concentration of from 0.1 to 20% by weight and preferably in a total concentration of from 0.5 to 10% by weight, respectively based on the total weight of the preparation.

The cosmetic preparations according to the invention may also advantageously, but not necessarily, comprise fillers which, for example, further improve the sensory and cosmetic properties of the formulations and for example bring about or enhance a velvety or silky feel on the skin. Advantageous fillers for the purposes of the present invention are starch and starch derivatives (such as, for example, tapioca starch, distarch phosphate, aluminum or sodium starch octenyl succinate and the like), pigments which have neither primarily a UV filter effect nor a coloring effect (such as, for example, boron nitride etc.) and/or Aerosils® (CAS No. 7631-86-9) and/or talc.

The aqueous phase of the preparations according to the invention may comprise one or more customary cosmetic auxiliaries, such as, e.g., alcohols, in particular those with a low C number, preferably ethanol and/or isopropanol, diols or polyols of low C number, and ethers thereof, preferably propylene glycol, 2-methyl-1,3-propanediol, glycerin, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes and in particular one or more thickeners, in particular, one or more thickeners which can advantageously be chosen from the group silicon dioxide, aluminum silicates, polysaccharides and derivatives thereof, e.g. hyaluronic acid, xanthan gum, carrageenans, hydroxypropylmethylcellulose, particularly advantageously from the group polyacrylates, preferably a polyacrylate from the group of so-called carbopols, for example, carbopols of the types 980, 981, 1382, 2984, 5984, either individually or in combination. Further thickeners that are advantageous according to the invention include those with the INCI designation acrylates/C10-30 alkyl acrylate crosspolymer (e.g., Pemulen TR1, Pemulen TR2, carbopol 1328 from NOVEON) and Aristoflex AVC (INCI: ammonium acryloyldimethyl taurate/VP copolymer).

Advantageously according to the invention the preparation according to the invention contains film formers. For the purposes of the present invention film formers are substances of differing composition that are characterized by the following property: if a film former is dissolved in water or other suitable solvents and the solution is then applied to the skin, after the solvent has evaporated a film is formed, which essentially serves to fix the light filters on the skin and thus to increase the water-resistance of the product.

It is particularly advantageous to select the film formers from the group of polymers based on polyvinyl pyrrolidone (PVP),

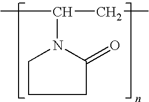

Particular preference is given to copolymers of polyvinyl pyrrolidone, for example, the PVP hexadecene copolymer and the PVP eicosene copolymer, which are available under the trade names Antaron V216 and Antaron V220 from GAF Chemicals Cooperation.

Also advantageous are further polymer film formers, such as, e.g., sodium polystyrene sulfonate, which is available from the National Starch and Chemical Corp. under the trade name Flexan 130, and/or polyisobutene, available from Rewo under the trade name Rewopal PIB1000. Other suitable polymers are, e.g., polyacrylamide (Seppigel 305), polyvinyl alcohols, PVP, PVP/VA copolymers, polyglycols, acrylate/octylacralymide copolymer (Dermacryl 79). Also advantageous is the use of hydrogenated castor oil dimer dilinoleate (CAS 646054-62-8, INCI Hydrogenated Castor Oil Dimer Dilinoleate), which is available from Kokyu Alcohol Kogyo under the name Risocast DA-H, or also PPG-3 benzyl ether myristate (CAS 403517-45-3), which is available under the trade name Crodamol STS from Croda Chemicals.

The oil phase of the preparation according to the invention is advantageously chosen from the group of polar oils, for example from the group of lecithins and of fatty acid triglycerides, namely the triglyceryl esters of saturated and/or unsaturated, branched and/or unbranched alkane carboxylic acids of a chain length of from 8 to 24, in particular 12 to 18, C atoms. The fatty acid triglycerides can, for example, advantageously be chosen from the group of synthetic, semi-synthetic and natural oils, such as, for example, cocoglyceride, olive oil, sunflower oil, jojoba oil, soya oil, peanut oil, rapeseed oil, almond oil, palm oil, coconut oil, castor oil, wheatgerm oil, grapeseed oil, thistle oil, evening primrose oil, macadamia nut oil, and the like.

Also advantageous according to the invention are, for example, natural waxes of animal and vegetable origin, such as, for example, beeswax and other insect waxes, and berry wax, shea butter and/or lanolin (wool wax).

Further advantageous polar oil components can also be chosen for the purposes of the present invention from the group of esters of saturated and/or unsaturated, branched and/or unbranched alkane carboxylic acids of a chain length of from 3 to 30 C atoms and saturated and/or unsaturated, branched and/or unbranched alcohols of a chain length of from 3 to 30 C atoms, and from the group of esters of aromatic carboxylic acids and saturated and/or unsaturated, branched and/or unbranched alcohols of a chain length of from 3 to 30 C atoms. Such ester oils can then advantageously be chosen from the group phenethyl benzoate, 2-phenylethyl benzoate, isopropyl lauroyl sarcosinate, phenyl trimethicone, cyclomethicone, dibutyladipate, octyl palmitate, octyl cocoate, octyl isostearate, octyl dodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and synthetic, semi-synthetic and natural mixtures of such esters, such as, for example, jojoba oil.

In addition, the oil phase can advantageously be chosen from the group of dialkyl ethers and dialkyl carbonates, for example dicaprylyl ether (Cetiol OE) and/or dicaprylyl carbonate, for example, that available under the trade name Cetiol CC from Cognis, being advantageous.

It is also preferred the oil component or components be from the group isoeicosane, neopentyl glycol diheptanoate, propylene glycol dicaprylate/dicaprate, caprylic/capric/diglyceryl succinate, butylene glycol dicaprylate/dicaprate, C₁₂₋₁₃ alkyl lactate, di-C₁₂₋₁₃ alkyl tartrate, triisostearin, dipentaerythrityl hexcaprylate/hexacaprate, propylene glycol monoisostearate, tricaprylin, dimethyl isosorbide. It is particularly advantageous if the oil phase of the formulations according to the invention has a content of C₁₂₋₁₅-alkyl benzoate or consists entirely thereof.

Advantageous oil components are also, for example, butyloctyl salicylate (for example that available under the trade name Hallbrite BHB from CP Hall), tridecylsalicylate (which is available from Sasol under the trade name Cosmacol ESI), C12-C15 alkylsalicylate (available from Alzo under the trade name Dermol NS), hexadecyl benzoate and butyloctyl benzoate and mixtures thereof (Hallstar AB) and/or diethylhexyl naphthalate (Hallbrite TQ or Corapan TQ from Symrise).

Any desired mixtures of such oil and wax components can also be used advantageously for the purposes of the present invention.

In addition, the oil phase can likewise advantageously also comprise nonpolar oils, for example those which are chosen from the group of branched and unbranched hydrocarbons and hydrocarbon waxes, in particular mineral oil, Vaseline (petrolatum), paraffin oil, squalane and squalene, polyolefins, hydrogenated polysiobutenes, C13-16 isoparaffin and isohexadecane. Among the polyolefins, polydecenes are the preferred substances.

The preparations according to the invention can also advantageously comprise one or more substances from the following group of siloxane elastomers, for example, in order to increase the water resistance and/or the light protection factor of the products:
(a) siloxane elastomers which contain the units R₂SiO and RSiO_{1.5} and/or R₃SiO_{0.5} and/or SiO₂,
   where the individual radicals R, in each case independently of one another, are hydrogen, C₁₋₂₄-alkyl (such as, for example, methyl, ethyl, propyl) or aryl (such as, for example, phenyl or tolyl), alkenyl (such as, for example, vinyl), and the weight ratio of the units R₂SiO to RSiO_{1.5} is chosen from the range of from 1:1 to 30:1;
(b) siloxane elastomers which are insoluble and swellable in silicone oil and which are obtainable by the addition reaction of an organopolysiloxane (1) which contains silicon-bonded hydrogen with an organopolysiloxane (2) which contains unsaturated aliphatic groups,
   where the quantitative amounts used are chosen such that the amount of hydrogen of the organopolysiloxane (1) or of the unsaturated aliphatic groups of the organopolysiloxane (2)
      is in the range from 1 to 20 mol % when the organopolysiloxane is non-cyclic and
      is in the range from 1 to 50 mol % when the organopolysiloxane is cyclic.

For the purposes of the present invention, the siloxane elastomer or elastomers are advantageously present in the form of spherical powders or in the form of gels.

Siloxane elastomers present in the form of spherical powders which are advantageous according to the invention are those with the INCI designation Dimethicone/Vinyl Dimethicone Crosspolymer, for example, that available from DOW CORNING under the trade names DOW CORNING 9506 Powder.

It is particularly preferred when the siloxane elastomer is used in combination with oils from hydrocarbons of animal and/or vegetable origin, synthetic oils, synthetic esters, synthetic ethers, or mixtures thereof.

Particularly advantageous preparations are also obtained when antioxidants are used as additives or active ingredients. According to the invention, the preparations advantageously comprise one or more antioxidants. Favorable, but nevertheless optional, antioxidants which may be used are all antioxidants customary or suitable for cosmetic applications.

For the purposes of the present invention, water-soluble antioxidants may be used particularly advantageously, such as, for example, vitamins, e.g. ascorbic acid and derivatives thereof.

Preferred antioxidants are also vitamin E and derivatives thereof, and vitamin A and derivatives thereof.

The amount of antioxidants (one or more compounds) in the preparations is preferably from 0.001 to 30% by weight, particularly preferably from 0.05 to 20% by weight, in particular from 0.1 to 10% by weight, based on the total weight of the preparation.

If vitamin E and/or derivatives thereof are the antioxidant or the antioxidants, it is advantageous to choose their respective concentrations from the range from 0.001 to 10% by weight, based on the total weight of the formulation.

If vitamin A or vitamin A derivatives, or carotenes or derivatives thereof are the antioxidant or the antioxidants, it is advantageous to choose their respective concentrations from the range from 0.001 to 10% by weight, based on the total weight of the formulation.

It is particularly advantageous when the cosmetic preparations according to the present invention comprise cosmetic active ingredients, preferred active ingredients being antioxidants which can protect the skin against oxidative stress.

Recipes according to the invention which comprise, for example, known anti-wrinkle active ingredients, such as flavone glycosides (in particular α-glycosylrutin), coenzyme Q10, retinol and ester, vitamin E and/or derivatives and the like are particularly advantageously suitable for the protection from esthetically unattractive changes in the skin, as arise, for example, during skin aging (such as, for example, dryness, roughness and formation of dryness wrinkles, itching, reduced refatting (e.g., after washing), visible vascular dilations (telangiectases, cuperosis), flaccidity and formation of wrinkles and lines, local hyperpigmentation, hypopigmentation and defective pigmentation (e.g., age spots), increased susceptibility to mechanical stress (e.g. cracking) and the like) and fatigued skin. In addition, they are advantageously suitable to counter the appearance of dry or rough skin.

The cosmetic preparations according to the invention can comprise cosmetic auxiliaries as are customarily used in such preparations, e.g., preservatives, preservative aids, complexing agents, bactericides, substances for preventing or increasing foaming, dyes, pigments which have a coloring effect, thickeners, moisturizing or humectant substances, fillers which improve the skin feel, fats, oils, waxes or other customary constituents of a cosmetic or dermatological formulation, such as alcohols, polyols, polymers, foam stabilizers, electrolytes, organic solvents or silicon derivatives.

Advantageous for the purposes of the present invention are preparations for the care of the skin: they can be used as cosmetic sunscreen, furthermore as a make-up product in decorative cosmetics.

According to their structure, cosmetic compositions for the purposes of the present invention can be used, e.g., as protective skin cream, day or night cream, etc. It is optionally possible and advantageous to use the compositions according to the invention as the basis for pharmaceutical formulations.

It is also advantageous for the purposes of the present invention to produce cosmetic preparations, the main purpose of which is not to protect against sunlight, but which nevertheless contain a content of UV protection substances. UVA or UVB filter substances are thus usually incorporated into day creams or make-up products, for example. UV protective substances, like antioxidants and, if desired, preservatives, also represent an effective protection of the preparations themselves against spoilage. Cosmetic preparations that are present in the form of a sunscreen agent are also favorable.

The use of the preparation according to the invention for protection against the aging of the skin (in particular against skin aging caused by UV) and as a sunscreen agent is particularly according to the invention.

The use of the preparations according to the invention for reducing or preventing fine lines and wrinkles is according to the invention.

The use of the preparations according to the invention for repairing the skin (in particular skin stressed by washing) is according to the invention.

It is advantageous according to the invention for the preparation according to the invention to have a pH value of from 5 to 8. This can be adjusted by means of the conventional acids, bases and buffer systems.

For application, the cosmetic preparations according to the invention are applied to the skin and/or the hair in sufficient quantities in the customary manner for cosmetics.

### EXAMPLES

The following examples are intended to illustrate the present invention without restricting it. Unless stated otherwise, all the quantities, proportions and percentages given are based on the weight and the total quantity or on the total weight of the preparations.

Day cream

| Example formula 1 | % by weight |
|---|---|
| Glyceryl stearate | 2.6 |
| Stearyl alcohol | 2 |
| PEG-40 stearate | 0.8 |
| White Vaseline | 3 |
| Cetyl alcohol | 4 |
| Shea butter | 3 |
| Titanium dioxide | 0.5 |
| Butyl methoxydibenzoylmethane | 2 |
| Linear silicone oil (dimethicone) | 0.5 |
| Tapioca starch | 1 |
| Methylparaben | 0.2 |
| Tocopherol acetate | 0.5 |
| Phenylbenzimidazole sulfonic acid sodium salt | 1.5 |
| Ethylhexyl methoxycinnamate | 7 |
| Phenoxyethanol | 0.4 |
| C₁₂₋₁₅-Alkyl benzoate | 3 |
| Propylparaben | 0.1 |
| Ethylparaben | 0.1 |
| Glucosyl glyceride | 3.5 |
| Glycerin | 8.7 |
| Polyacrylic acid, Na salt | 0.1 |
| Carrageenan | 0.2 |
| Ethylhexylglycerin | 0.5 |
| Na₃EDTA | 0.2 |
| Benzyl benzoate | 0.0025 |
| Caprylic/capric triglyceride | 1 |
| Methylpropanediol | 4 |
| Water 100% | ad 100 |

Night cream

| Example formula 2 | % by weight |
|---|---|
| Tapioca starch | 2 |
| Cetyl alcohol | 2 |
| Glyceryl stearate citrate | 2 |
| Shea butter | 4 |
| Hydrogenated cocoglycerides | 3 |
| Ethylhexyl methoxycinnamate | 5 |
| Phenoxy ethanol | 0.4 |
| Linear silicone oil (dimethicone) | 1.5 |
| Cyclic silicone oil (cyclomethicone) | 5 |
| Caprylyl carbonate | 5 |
| Nylon-12 | 1.5 |
| Tocopheryl acetate | 1 |
| Squalane | 2 |
| Avocado oil | 1 |
| Carrageenan | 0.1 |
| Polyacrylic acid, Na salt | 0.1 |
| Methylpropanediol | 4 |
| Methylparaben | 0.15 |
| Glucosyl glyceride | 7.5 |
| Glycerin | 8.7 |
| Na₃EDTA | 0.2 |
| Hydroxycitronellal | 0.0004 |
| Butylene glycol + iodopropynyl butylcarbamate | 0.09 |
| C₁₂₋₁₅ Alkylbenzoate | 3 |
| Acrylates/C10-30 Alkyl Acrylate Crosspolymer | 0.1 |
| 1,2-Hexanediol | 0.5 |
| Water | ad 100 |

Eye Care Cream

| Example formula 3 | % by weight |
|---|---|
| Glyceryl stearate | 2.6 |
| Hydrogenated vegetable oil | 1.5 |
| PEG-40 Stearate | 0.8 |
| White Vaseline | 1.5 |
| Cetyl stearyl alcohol | 1.5 |
| Shea butter | 3 |
| Titanium dioxide | 5 |
| Linear silicone oil (dimethicone) | 2.5 |
| Tapioca starch | 1.5 |
| Methylparaben | 0.21 |
| Tocopherol acetate | 0.5 |
| Nylon-12 | 1.5 |
| Phenoxyethanol | 0.4 |
| C₁₂₋₁₅ Alkyl benzoate | 2 |
| Cyclic silicone oil (cyclomethicone) | 4 |
| Propylparaben | 0.05 |
| Ethylparaben | 0.11 |
| Methylpropanediol | 2 |
| Glucosyl glyceride | 10 |
| Octyldodecanol | 2 |
| Glycerin | 6.5 |
| Polyacrylic acid, Na salt | 0.1 |
| Caprylyl glycol | 0.25 |
| Na₃EDTA | 0.2 |
| Hydroxyisohexyl 3-cyclohexene carboxaldehyde | 0.0025 |
| Caprylic/capric triglyceride | 2 |
| Water | ad 100 |

Body Serum

| Example formula 4 | % by weight |
|---|---|
| Glyceryl stearate | 2 |
| Squalane | 1.5 |
| PEG-40 Stearate | 1 |
| Medicinal white mineral oils (mixture of various | 15.1 |
| viscosities) | |
| Stearyl alcohol | 3.5 |
| Hydrogenated cocoglycerides | 3 |
| Linear silicone oil (dimethicone) | 0.5 |
| Myristyl myristate | 1.5 |
| Methylparaben | 0.3 |
| Tocopherol acetate | 0.5 |
| Nylon-12 | 1.5 |
| Phenoxyethanol | 0.5 |
| C₁₂₋₁₅ Alkyl benzoate | 2 |
| Cyclic silicone oil (cyclomethicone) | 4 |
| Propylparaben | 0.1 |
| Ethylparaben | 0.11 |
| Methylpropanediol | 2 |
| Glucosyl glyceride | 5.5 |
| Octyldodecanol | 2 |
| Butyl methoxydibenzoylmethane | 0.5 |
| Octocrylene | 6 |
| Glycerin | 10 |
| Polyacrylic acid, Na salt | 0.02 |
| Caprylyl glycol | 0.25 |
| Na₃EDTA | 0.2 |
| Hexylcinnamal + hexylsalicylate | 0.0035 |
| Caprylic/capric triglyceride | 2 |
| Water 100% | ad 100 |

O/W Emulsion

| Example formula 5 | % by weight |
|---|---|
| Caprylic acid/capric acid triglycerides | 3 |
| Sorbitan stearate | 1 |
| Stearyl alcohol | 1 |
| Polyglyceryl-3-methylglucose distearate | 3 |
| 2,4-Bis-[5-1(dimethylpropyl)benzoxazol-2-yl-(4-phenyl)-imino]-6- | 1 |
| (2-ethylhexyl)-imino-1,3,5-triazine (CAS no. 288254-16-0) | |
| Butyl methoxydibenzoylmethane | 1 |
| Ethylhexyl methoxycinnamate | 3 |
| Ethylhexyl triazone | 1 |
| Linear silicone oil (dimethicone) | 2 |
| Cyclic silicone oil (cyclomethicone) | 4 |
| C₁₂₋₁₅ Alkyl benzoate | 2 |
| Dicaprylyl ether | 2 |
| Carrageenan | 0.1 |
| Cross-linked alkyl acrylate (Alkylacrylate Crosspolymer) | 0.1 |
| Glucosyl glyceride | 3.5 |
| Glycerin | 8 |
| Methylheptenone | 0.001 |
| Na₃EDTA | 0.2 |
| Licochalcone A | 0.02 |
| Ethylhexylglycerin | 0.4 |
| Citric acid, sodium salt (pH 6.5) | q.s. |
| Water | ad 100 |

Sunblock Cream

| Example formula 6 | % by weight |
|---|---|
| Glyceryl stearate | 2.5 |
| PEG-40 Stearate | 1 |
| Cetyl alcohol | 2.5 |
| C₁₂₋₁₅ Alkyl benzoate | 2 |
| Hydrogenated cocoglycerides | 2 |
| Octyldodecanol | 2 |
| Cyclic silicone oil (cyclomethicone) | 2 |
| Beeswax | 1 |
| Ethylhexyl methoxycinnamate | 6 |
| Phenylbenzimidazole sulfonic acid | 2 |
| Sodium ascorbyl phosphate | 0.1 |
| Methyl propanediol | 2 |
| Diazolidinyl urea | 0.1 |
| Carrageenan | 0.1 |
| Polyacrylic acid, Na salt | 0.1 |
| Benzyl salicylate | 0.2 |
| Glycerin | 7 |
| Glucosyl glyceride | 3.5 |
| Na₃EDTA | 0.2 |
| 2,4-Bis-{[4-(2-ethylhexyloxy)-2-hydroxy]-phenyl}-6-4- | 2 |
| methoxyphenyl)-1,3,5-triazine (Tinosorb ®S) | |
| Aqueous sodium hydroxide (pH 6.8) | q.s. |
| Water | ad 100 |

Examples 7-10

| O/W Emulsions | 7 | 8 | 9 | 10 |
|---|---|---|---|---|
| Perfume | 0.3 | | 0.3 | |
| Na₃HEDTA | 0.5 | 1 | 0.5 | 1 |
| Glycerin | 10 | 7.5 | 7.5 | 25 |
| Glyceryl stearate citrate | 2 | 2.5 | | |
| PEG-40 Stearate | | | 1.0 | 0.7 |
| Glyceryl stearate SE | | | 2.5 | 2.5 |
| Stearyl alcohol | 2 | 3 | 1 | 1.5 |
| Cetyl alcohol | 2 | | 1 | |
| Polyacrylic acid, Na salt | 0.1 | 0.25 | 0.3 | 0.1 |
| Acrylate/C10-30 Alkylacrylate Crosspolymer | 0.2 | 0.3 | 0.1 | 0.2 |
| Chondrus crispus | 0.2 | | | |
| Myristyl myristate | | | 1 | 1 |
| C₁₂₋₁₅ Alkyl benzoate | 2 | 4 | 2 | 4 |
| Octyldodecanol | 2 | | | 2 |
| Caprylic acid/capric acid triglycerides | 1 | 2 | 1 | 2 |
| Medicinal white mineral oil | 3 | | 2 | |
| Butyrospermum parkii | | 3 | 3 | |
| Linear silicone oil (dimethicone) | 1 | 2 | 1 | 3 |
| Cyclic silicone oil (cyclomethicone) | 3 | 4 | 1.6 | 3 |
| Methylparaben | 0.4 | 0.3 | | 0.2 |
| Phenoxyethanol | 0.4 | 0.6 | 0.4 | 0.5 |
| Diazolidinyl urea | | | 0.2 | |
| Ethylhexylglycerin | 0.3 | | | |
| Capryl glycol | 0.25 | | | |
| 1,2-Hexanediol | | 0.5 | | 0.5 |
| Methylpropanediol | 3 | | 2 | 2 |
| Butyl methoxydibenzoylmethane | | 2 | | 3 |
| Ethylhexyl methoxycinnamate | | 5 | | 7 |
| Phenylbenzimidazole sulfonic acid | | 2 | | 2 |
| Glucosyl glyceride | 11.5 | 7.5 | 26 | 20 |
| Nylon-12 | | 1.5 | 1.5 | |
| Titanium dioxide hydrophilically coated | | | 5 | |
| Titanium dioxide hydrophobically coated | 0.5 | 2 | 1.5 | 0.5 |
| Tapioca starch | 2 | | 1.5 | 3 |
| Talc | 1.5 | 2 | 0.5 | |
| Tocopherol acetate | 0.5 | 1 | 1 | 1.5 |
| d-Limonene + ethyl linalool + sweet orange oil | 0.002 | 0.01 | 0.25 | 0.0035 |
| Water | Ad 100 | Ad 100 | Ad 100 | Ad 100 |

1.-6. (canceled)

7. A cosmetic preparation, wherein the preparation comprises
(a) one or more perfume substances selected from 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic diester, alpha-amylcinnamaldehyde, alpha-methylionone, amyl C butylphenyl methylpropional cinnamal, amyl salicylate, amyl cinnamyl alcohol, anisic alcohol, benzoin, benzyl alcohol, benzyl benzoate, benzyl cinnamate, benzyl salicylate, bergamot oil, bitter orange oil, butylphenyl methylpropional, cardamom oil, cedrol, cinnamal, cinnamyl alcohol, citronellyl methylcrotonate, lemon oil, coumarin, diethyl succinate, d-limonene, ethyl linalool, eugenol, evernia furfuracea extract, evernia prunastri extract, farnesol, guaiacwood oil, hexyl cinnamal, hexyl salicylate, hydroxycitronellal, hydroxyisohexyl 3-cyclohexene carboxaldehyde, lavender oil, lime oil, linalyl acetate, mandarin oil, menthyl PCA, methyl heptenone, myristica oil, oil of rosemary, sweet orange oil, terpineol, tonka bean oil, triethylcitrate, and vanillin; and
(b) one or more glucosyl glycerides.

8. The preparation of claim 7, wherein the preparation comprises from 0.01% to 10% by weight of (b), based on a total weight of the preparation.

9. The preparation of claim 8, wherein the preparation comprises from 0.05% to 6% by weight of (b).

10. The preparation of claim 8, wherein the preparation comprises from 0.1% to 3% by weight of (b).

11. The preparation of claim 7, wherein the preparation comprises from 0.005% to 0.5% by weight of (a), based on a total weight of the preparation.

12. The preparation of claim 7, wherein the preparation is free of (2-hydroxyethyl)urea.

13. The preparation of claim 7, wherein the preparation is present as at least one of a gel, an emulsion, and a dispersion.

14. The preparation of claim 7, wherein the preparation is present as an O/W emulsion.

15. The preparation of claim 14, wherein the preparation comprises one or more O/W emulsifiers selected from glyceryl stearate citrate, glyceryl stearate, cetearyl glucoside, stearic acid and salts thereof, polyglyceryl-3-methylglycose distearate, ceteareth-20, PEG-40 stearate, PEG-100 stearate, and sodium cetearyl sulfate.

16. The preparation of claim 7, wherein the preparation further comprises one or more further UV filter substances.

17. The preparation of claim 16, wherein the one or more UV filter substances comprise at least one of homomethyl salicylate, 2-ethylhexyl 2-cyano-3,3-diphenylacrylate, terephthalidene dicamphor sulfonic acid, 2-ethylhexyl-2-hydroxybenzoate, an ester of cinnamic acid, a phenylene-1,4-bis-(2-benzimidazyl)-3,3′-5,5′-tetrasulfonic acid salt, a 2-phenylbenzimidazol-5-sulfonic acid salt, a 3-(4-(2,2-bis-ethoxycarbonylvinyl)-phenoxy)propenyl)-methoxysilane/dimethyl-siloxane copolymer, 2,4-bis-{[4-(2-ethylhexyloxy)-2-hydroxy]-phenyl}-6-(4-methoxyphenyl)-1,3,5-triazine, dioctylbutylamidotriazone, 2,4-bis-[5-1(dimethylpropyl)benzoxazol-2-yl-(4-phenyl)-imino]-6-(2-ethylhexyl)-imino-1,3,5-triazine, 2,4,6-tribiphenyl-4-yl-1,3,5-triazine; merocyanine, titanium dioxide, and zinc oxide.

18. The preparation of claim 17, wherein the preparation comprises from 0.01% to 40% by weight of the one or more UV filter substances, based on a total weight of the preparation.

19. The preparation of claim 18, wherein the preparation comprises from 1% to 20% by weight of the one or more UV filter substances.

20. The preparation of claim 7, wherein the preparation further comprises one or more substances selected from folic acid, D-biotin, coenzyme Q10, alpha-glucosylrutin, carnitine, natural and/or synthetic isoflavonoids, genistein, flavonoides, carotenoids, creatine, creatinine, taurine, urea, ascorbic acid and derivatives thereof, oxygen, tocopherol and esters thereof, dihydroxyacetone, long chain and short chain hyaluronic acids, 8-hexadecene-1,16-dicarboxylic acid, and licochalcone A.

21. The preparation of claim 7, wherein (a) comprises at least one of hydroxycitronellal, hydroxyisohexyl 3-cyclohexene carboxaldehyde, hexylcinnamal, hexylsalicylate, methylheptenone, d-limonene, ethyl linalool, and sweet orange oil.

22. A cosmetic preparation, wherein the preparation comprises, based on a total weight of the preparation,
from 0.005% to 0.5% by weight, of one or more perfume substances selected from 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic diester, alpha-amylcinnamaldehyde, alpha-methylionone, amyl C butylphenyl methylpropional cinnamal, amyl salicylate, amyl cinnamyl alcohol, anisic alcohol, benzoin, benzyl alcohol, benzyl benzoate, benzyl cinnamate, benzyl salicylate, bergamot oil, bitter orange oil, butylphenyl methylpropional, cardamom oil, cedrol, cinnamal, cinnamyl alcohol, citronellyl methylcrotonate, lemon oil, coumarin, diethyl succinate, d-limonene, ethyl linalool, eugenol, evernia furfuracea extract, evernia prunastri extract, farnesol, guaiacwood oil, hexyl cinnamal, hexyl salicylate, hydroxycitronellal, hydroxyisohexyl 3-cyclohexene carboxaldehyde, lavender oil, lime oil, linalyl acetate, mandarin oil, menthyl PCA, methyl heptenone, myristica oil, oil of rosemary, sweet orange oil, terpineol, tonka bean oil, triethylcitrate, and vanillin; and
from 0.05% to 6% by weight of one or more glucosyl glycerides.

23. The preparation of claim 22, wherein the preparation comprises from 0.1% to 3% by weight of the one or more glucosyl glycerides.

24. The preparation of claim 22, wherein the preparation is present as an O/W emulsion.

25. The preparation of claim 22, wherein the preparation further comprises or more further UV filter substances.

26. The preparation of claim 22, wherein the preparation further comprises one or more substances selected from folic acid, D-biotin, coenzyme Q10, alpha-glucosylrutin, carnitine, natural and/or synthetic isoflavonoids, genistein, flavonoides, carotenoids, creatine, creatinine, taurine, urea, ascorbic acid and derivatives thereof, oxygen, tocopherol and esters thereof, dihydroxyacetone, long chain and short chain hyaluronic acids, 8-hexadecene-1,16-dicarboxylic acid, and licochalcone A.
